(19) United States
(12) Patent Application Publication
(10) Pub. No.: US 2003/0147853 A1
(43) Pub. Date: Aug. 7, 2003

(54) Recombinant adeno-associated virus-mediated gene transfer via retroductal infusion of virions

(76) Inventors: Alan McClelland, Danville, CA (US); Roland Scollay, Melbourne (AU)

Correspondence Address: COOLEY GODWARD LLP (R & P), FIVE PALO ALTO SQUARE, 3000 EL CAMINO REAL, PALO ALTO, CA, 94306-2155, US

(21) Appl. No.: 10/100,235

(22) Filed: Mar. 14, 2002

Related U.S. Application Data
(60) Provisional application No. 60/275,908, filed on Mar. 14, 2001.

(51) Int. Cl. A61K 48/00; C12N 15/861; C12N 7/00

(52) U.S. Cl. 424/93.2; 435/456; 435/235.1

(57) ABSTRACT

Methods for introducing recombinant adeno-associated virus (rAAV) virions into a cell or cells of a secretory gland are described. Recombinant AAV virions containing a heterologous gene are introduced into a duct of a secretory gland resulting in transduction of one or more secretory gland cells. Once a secretory gland cell is transduced by the rAAV virion, the heterologous gene is expressed and the expression product is then secreted. Exemplary examples of secretory glands are the liver, the submandibular gland, the parotid gland, and the sublingual gland. Using the methods of the invention, therapeutic levels of a protein are achieved. Methods for treating hemophilia are also disclosed.

### CROSS REFERENCE TO RELATED APPLICATIONS

This application claims the benefit under 35 U.S.C. § 119(e)(1) of Provisional Application Ser. No. 60/275,908, filed on Mar. 14, 2001.

### FIELD OF THE INVENTION

The present invention relates to methods of delivering recombinant adeno-associated virus (rAAV) virions to a mammalian subject. More specifically, the invention relates to methods in which rAAV virions are administered to the duct of a secretory gland of a mammalian subject, including a human, to deliver therapeutic proteins.

### BACKGROUND OF THE INVENTION

Scientists are continually discovering genes that are associated with human diseases such as diabetes, hemophilia, and cancer. Research efforts have also uncovered genes, such as erythropoietin, that are not associated with genetic disorders but instead code for proteins that can be used to treat numerous diseases. Despite significant progress in the effort to identify and isolate genes, however, a major obstacle facing the biopharmaceutical industry is how to safely and persistently deliver therapeutically effective quantities of gene products to patients.

Generally, the protein products of these genes are synthesized in cultured bacterial, yeast, insect, mammalian, or other cells and delivered to patients by direct injection. Injection of recombinant proteins has been successful but suffers from several drawbacks. For example, patients often require weekly, and sometimes daily, injections in order to maintain the necessary levels of the protein in the blood stream. Even then, the concentration of protein is not maintained at physiological levels—the level of the protein is usually abnormally high immediately following the injection, and far below optimal levels prior to the injection. Additionally, injected delivery of recombinant protein often cannot deliver the protein to the target cells, tissues, or organs in the body. And, if the protein reaches its target, it is often diluted to non-therapeutic levels. Furthermore, the method is inconvenient and severely restricts the patient's lifestyle. The adverse impact on lifestyle is especially significant when the patient is a child.

These shortcomings have led to the development of gene therapy methods for delivering sustained levels of specific proteins into patients. These methods are designed to allow clinicians to introduce deoxyribonucleic acid (DNA) coding for a heterologous nucleic acid molecule (HNA) directly into a patient (in vivo gene therapy) or into cells isolated from a patient or a donor (ex vivo gene therapy), which are subsequently returned to the patient. The introduced DNA then directs the patient's own cells or grafted cells to produce the desired protein product. Gene delivery, therefore, obviates the need for frequent injections. Gene therapy may also allow clinicians to select specific organs or cellular targets (e.g., muscle, blood cells, brain cells, etc.) for therapy.

DNA may be introduced into a patient's cells in several ways. There are transfection methods, including chemical methods such as calcium phosphate precipitation and liposome-mediated transfection, and physical methods such as electroporation. In general, transfection methods are not suitable for in vivo gene delivery. There are also methods that use recombinant viruses. Current viral-mediated gene delivery vectors include those based on retrovirus, adenovirus, herpes virus, pox virus, and adeno-associated virus (AAV). Like the retroviruses, and unlike adenovirus, AAV has the ability to integrate its genome into a host cell chromosome.

### Adeno-Associated Virus-Mediated Gene Therapy

AAV, a parvovirus belonging to the genus Dependovirus, has several attractive features not found in other viruses. For example, AAV can infect a wide range of host cells, including non-dividing cells. Furthermore, AAV can infect cells from different species. Importantly, AAV has not been associated with any human or animal disease, and does not appear to alter the physiological properties of the host cell upon integration. Finally, AAV is stable at a wide range of physical and chemical conditions, which lends itself to production, storage, and transportation requirements.

There are six known AAV serotypes, AAV-1 through AAV-6. Of those six serotypes, AAV-2 is the best characterized. For instance, AAV-2 has been used in a broad array of transduction experiments, and has been shown to transduce many different tissue types.

The AAV genome, a linear, single-stranded DNA molecule containing approximately 4700 nucleotides (the AAV-2 genome consists of 4681 nucleotides), generally comprises an internal non-repeating segment flanked on each end by inverted terminal repeats (ITRs). The ITRs are approximately 145 nucleotides in length (AAV-1 has ITRs of 143 nucleotides) and have multiple functions, including serving as origins of replication, and as packaging signals for the viral genome.

The internal non-repeated portion of the genome includes two large open reading frames (ORFs), known as the AAV replication (rep) and capsid (cap) regions. These ORFs encode replication and capsid gene products, respectively: replication and capsid gene products (i.e., proteins) allow for the replication, assembly, and packaging of a complete AAV virion. More specifically, a family of at least four viral proteins are expressed from the AAV rep region: Rep 78, Rep 68, Rep 52, and Rep 40, all of which are named for their apparent molecular weights. The AAV cap region encodes at least three proteins: VP1, VP2, and VP3.

AAV is a helper-dependent virus, requiring co-infection with a helper virus (e.g., adenovirus, herpesvirus, or vaccinia virus) in order to form functionally complete AAV virions. In the absence of co-infection with a helper virus, AAV establishes a latent state in which the viral genome inserts into a host cell chromosome or exists in an episomal form, but infectious virions are not produced. Subsequent infection by a helper virus “rescues” the integrated genome, allowing it to be replicated and packaged into viral capsids, thereby reconstituting the infectious virion. While AAV can infect cells from different species, the helper virus must be of the same species as the host cell. Thus, for example, human AAV will replicate in canine cells that have been co-infected with a canine adenovirus.

To produce recombinant AAV (rAAV) virions containing the HNA, a suitable host cell line is transfected with an AAV vector containing the HNA, but lacking rep and cap. The host cell is then infected with wild-type (wt) AAV and a suitable helper virus to form rAAV virions. Alternatively, wt AAV genes (known as helper function genes, comprising rep and cap) and helper virus function genes (known as accessory function genes) can be provided in one or more plasmids, thereby eliminating the need for wt AAV and helper virus in the production of rAAV virions. The helper and accessory function gene products are expressed in the host cell where they act in trans on the rAAV vector containing the heterologous gene. The heterologous gene is then replicated and packaged as though it were a wt AAV genome, forming a recombinant AAV virion. When a patient's cells are transduced with the resulting rAAV virion, the HNA enters and is expressed in the patient's cells. Because the patient's cells lack the rep and cap genes, as well as the accessory function genes, the rAAV virion cannot further replicate and package its genomes. Moroever, without a source of rep and cap genes, wt AAV virions cannot be formed in the patient's cells.

### AAV Delivery Limitations

Systemic (e.g., intravascular) administration of AAV can lead to unwanted biodistribution. For example, although a desired outcome from systemic AAV administration may be the transduction of the liver, such an approach can lead to the transduction of other tissues, which may limit therapeutic effectiveness and/or require higher doses of vector to achieve a therapeutic effect. Furthermore, it cannot be discounted that germline transmission may occur as a consequence of systemic administration, although this may be predicated on the route of administration (Arruda et al., (2001) Mol Ther. 4:586-592).

Many if not most current AAV delivery methods require that the patient be subject to an invasive procedure. In general, the more desirous it is to target AAV to a specific organ or tissue (e.g., to limit biodistribution), the more invasive the procedure necessary to achieve target specificity. For example, to target the liver specifically, current procedures rely on conducting a laparotomy to inject AAV directly into the liver, or intravascular administration requiring, at a minimum, a surgical incision in the leg to gain access to the femoral artery for subsequent catheter delivery to the hepatic artery. Although effective, such procedures can have unwanted effects, such as significant post-operative pain, recovery time, risk of nosocomial infection and the like.

It would be an advancement in the art, therefore, to develop non- or minimally invasive procedures for delivering AAV virions that circumvent exposure to the patient's bloodstream, thereby potentially limiting unwanted biodistribution and reducing the required dose of rAAV virions necessary to elicit a therapeutic effect or a desired response. Such methods are disclosed herein.

### SUMMARY OF THE INVENTION

In accordance with the present invention, methods and vectors for use are provided for the efficient delivery of heterologous nucleic acid molecules (e.g., encoding genes that express proteins, anti-sense RNA, and ribozymes) to a secretory gland of a mammal, using rAAV virions. The methods provide for the introduction of rAAV virions into the duct of a secretory organ, the transduction of the associated secretory gland cells, and the long-term expression of a gene product.

In a preferred embodiment, heterologous genes encode secretory proteins, which are delivered to the cells of a secretory gland by rAAV virions. Preferably, the rAAV virions are administered at a dose from about 1×10⁹ viral genomes (vg)/mammal to about 1 ×10¹¹ vg/mammal. Once expressed, the proteins are secreted from the cell, preferably into the bloodstream, at levels sufficient to achieve a therapeutic effect. In a preferred embodiment, the mammal is a human.

In one embodiment of the invention, the secretory gland is a salivary gland, preferably a submandibular gland. In another embodiment, the secretory gland is a liver. In one aspect of the invention, the rAAV virions are delivered to the hepatic duct. In another aspect, the rAAV virions are delivered to the common bile duct.

Preferably, rAAV virions are delivered to the duct of a secretory gland by means of retrograde ductal administration. When the secretory gland is a liver, a preferred way of delivering rAAV virions is by endoscopic retrograde cholangiopancreatography.

It is an object of the present invention to deliver rAAV virions containing a gene encoding a blood coagulation protein which, when expressed in secretory gland cells, improves the blood-clotting efficiency of a mammal having hemophilia. Preferably, the mammal is a human. In one aspect of the invention, the mammal has hemophilia A. In another aspect, the mammal has hemophilia B. Preferably, the rAAV virions are delivered to the secretory gland by retroductal administration.

The blood coagulation protein gene can be expressed in the secretory gland by means of a tissue-specific promoter. In one aspect, the tissue-specific promoter is a liver-specific promoter, preferably a human alpha 1-antitrypsin (HAAT) promoter. In an especially preferred embodiment, the HAAT promoter is operably linked to an apolipoprotein E hepatic control region.

In one embodiment of the invention, the blood coagulation protein is Factor IX (F.IX), preferably human F.IX (hF.IX). In another embodiment, the blood coagulation protein is Factor VIII (F.VIII), preferably human F.VIII (hF.VIII).

### BRIEF DESCRIPTION OF THE DRAWINGS

FIG. 1 depicts circulating plasma hF.IX in nanograms per milliliter (ng/mL) as described in Example 2. Retrograde submandibular gland ductal injection was conducted and injection volume was 50 μL of rAAV-2-hF.IX virions for each dose: low dose=1×10⁹ viral genomes (vg)/mouse, medium dose=1×10¹⁰ vg/mouse, and high dose=1 ×10¹¹ vg/mouse (n=6 mice per dose).

FIG. 2 depicts circulating plasma hF.IX in ng/mL as described in Example 3. Retrograde hepatic ductal injection was conducted and injection volume was 250 μL of rAAV-2-hF.IX virions for each dose: low dose=1×10⁹ vg/mouse, medium dose=1×10¹⁰ vg/mouse, and high dose=1×10¹¹ vg/mouse (n=6 mice per dose).

FIG. 3 compares circulating plasma levels of hF.IX levels (in ng/mL) from portal vein injection and retrograde hepatic ductal injection of rAAV-2-hF.IX virions as described in Example 4. Injection volume was 250 μL of rAAV-2-hF.IX virions for both routes of administration (low dose=1×10⁹ vg/mouse, medium dose=1×10¹⁰ vg/mouse, and high dose=1×10¹ vg/mouse) (n=6 mice per dose for hepatic duct injection and n=3 mice per dose for portal vein injection).

### DETAILED DESCRIPTION OF THE INVENTION

The present invention embraces the use of a recombinant adeno-associated virus (rAAV) virion to deliver a heterologous nucleic acid (HNA) to a cell of a secretory gland of a mammalian subject. Once delivered, the HNA is transcribed and, in the case where the HNA comprises a gene, the transcription product is then translated into a protein and the protein is secreted from the cell.

In the context of the present invention, a “recombinant AAV virion” or “rAAV virion” is an infectious virus composed of an AAV protein shell (i.e., a capsid) encapsulating a “recombinant AAV (rAAV) vector,” the rAAV vector defined herein as comprising the HNA and one or more AAV inverted terminal repeats (ITRs). AAV vectors can be constructed using recombinant techniques that are known in the art and include one or more HNAs flanked by functional ITRs. The ITRs of the rAAV vector need not be the wild-type nucleotide sequences, and may be altered, e.g., by the insertion, deletion, or substitution of nucleotides, so long as the sequences provide for proper function, i.e., rescue, replication, and packaging of the AAV genome.

Recombinant AAV virions may be produced using a variety of techniques that are well known in the art. For example, the skilled artisan can use wt AAV and helper viruses to provide the necessary replicative functions for producing rAAV virions (see, e.g., U.S. Pat. No. 5,139,941, herein incorporated by reference). Alternatively, a plasmid, containing helper function genes, in combination with infection by one of the well-known helper viruses can be used as the source of replicative functions (see e.g., U.S. Pat. No. 5,622,856, herein incorporated by reference; U.S. Pat. No. 5,139,941, supra). Similarly, the skilled artisan can make use of a plasmid, containing accessory function genes, in combination with infection by wt AAV to provide the necessary replicative functions. As is familiar to one of skill in the art, these three approaches, when used in combination with a rAAV vector, are each sufficient to produce rAAV virions. Other approaches, well known in the art, can also be employed by the skilled artisan to produce rAAV virions.

In a preferred embodiment of the present invention, the triple transfection method (described in detail in U.S. Pat. No. 6,001,650, the entirety of which is incorporated by reference) is used to produce rAAV virions because this method does not require the use of an infectious helper virus, enabling rAAV virions to be produced without any detectable helper virus present. This is accomplished by use of three vectors for rAAV virion production: an AAV helper function vector, an accessory function vector, and a rAAV vector. One of skill in the art will appreciate, however, that the nucleic acid sequences encoded by these vectors can be provided on two or more vectors in various combinations. As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors.

The AAV helper function vector encodes the “AAV helper function” sequences (i.e., rep and cap), which function in trans for productive AAV replication and encapsidation. Preferably, the AAV helper function vector supports efficient AAV vector production without generating any detectable wt AAV virions (i.e., AAV virions containing functional rep and cap genes). An example of such a vector, pHLP19 is described in U.S. Pat. No. 6,001,650, supra, and in Example 1, infra. The rep and cap genes of the AAV helper function vector can be derived from any of the known AAV serotypes. For example, the AAV helper function vector may have a rep gene derived from AAV-2 and a cap gene derived from AAV-6; one of skill in the art will recognize that other rep and cap gene combinations are possible, the defining feature being the ability to support rAAV virion production.

The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the well-known helper viruses such as adenovirus, herpesvirus (other than herpes simplex virus type-1), and vaccinia virus. In a preferred embodiment, the accessory function plasmid pLadeno5 is used (details regarding pLadeno5 are described in U.S. Pat. No. 6,004,797, the entirety of which is hereby incorporated by reference). This plasmid provides a complete set of adenovirus accessory functions for AAV vector production, but lacks the components necessary to form replication-competent adenovirus.

The “rAAV vector” can be a vector derived from any AAV serotype, including without limitation, AAV-1, AAV-2, AAV-3A, AAV-3B, AAV-4, AAV-5, AAV-6, etc. AAV vectors can have one or more of the wt AAV genes deleted in whole or in part, i.e., the rep and/or cap genes, but retain at least one functional flanking ITR sequence, as necessary for the rescue, replication, and packaging of the AAV virion. Thus, an AAV vector is defined herein to include at least those sequences required in cis for viral replication and packaging (e.g., functional ITRs). The ITRs need not be the wild-type nucleotide sequences, and may be altered, e.g., by the insertion, deletion, or substitution of nucleotides, so long as the sequences provide for functional rescue, replication, and packaging. AAV vectors can be constructed using recombinant techniques that are known in the art to include one or more HNAs flanked with functional AAV ITRs, the incorporation of the HNA defining a “rAAV vector.”

The HNA, that is, the “heterologous nucleic acid,” comprises nucleic acid sequences joined together that are otherwise not found together in nature, this concept defining the term “heterologous.” To illustrate the point, an example of an HNA is a gene flanked by nucleotide sequences not found in association with that gene in nature. Another example of an HNA is a gene that itself is not found in nature (e.g., synthetic sequences having codons different from the native gene). Allelic variation or naturally occurring mutational events do not give rise to HNAs, as used herein. An HNA can comprise an anti-sense RNA molecule, a ribozyme, or a gene encoding a polypeptide.

The HNA is operably linked to a heterologous promoter (constitutive, cell-specific, or inducible) such that the HNA is capable of being transcribed in the patient's target cells under appropriate or desirable conditions. By “operably linked” is meant an arrangement of elements wherein the components so described are configured so as to perform their usual function. Thus, control sequences operably linked to a coding sequence are capable of effecting the transcription of the coding sequence. The control sequences need not be contiguous with the coding sequence, so long as they function to direct the transcription thereof. Thus, for example, intervening untranslated yet transcribed sequences can be present between a promoter sequence and the coding sequence and the promoter sequence can still be considered “operably linked” to the coding sequence.

Numerous examples of constitutive, cell-specific, and inducible promoters are known in the art, and one of skill could readily select a promoter for a specific intended use, e.g., the selection of the liver-specific human alpha-1 antitrypsin promoter for liver cell-specific expression or the selection of the salivary gland-specific salivary alpha-amylase promoter for salivary gland-specific expression, the selection of the constitutive CMV promoter for strong levels of continuous or near-continuous expression, or the selection of the inducible ecdysone promoter for induced expression. Induced expression allows the skilled artisan to control the amount of protein that is synthesized. In this manner, it is possible to vary the concentration of therapeutic product. Other examples of well known inducible promoters are: steroid promoters (e.g., estrogen and androgen promoters) and metallothionein promoters.

Heterologous nucleic acid expression can be “enhanced” by way of an “enhancer element.” By “enhancer element” is meant a DNA sequence (i.e., a cis-acting element) that, when bound by a transcription factor, increases expression of a gene relative to expression from a promoter alone. There are many enhancer elements known in the art, and the skilled artisan can readily select an enhancer element for a specific purpose. An example of an enhancer element useful for increasing gene expression in the liver is the apolipoprotein E hepatic control region (described in Schachter et al. (1993) J Lipid Res 34:1699 -1707 and in Example 1, infra).

The invention embraces rAAV virions comprising HNAs coding for one or more anti-sense RNA molecules. Antisense RNA molecules suitable for use with the present invention in cancer anti-sense therapy or treatment of viral diseases have been described in the art. See, e.g., Han et al., (1991) Proc. Natl. Acad. Sci. USA 88:4313-4317; Uhlmann et al., (1990) Chem. Rev. 90:543-584; Helene et al., (1990) Biochim. Biophys. Acta. 1049:99-125; Agarawal et al., (1988) Proc. Natl. Acad. Sci. USA 85:7079-7083; and Heikkila et al., (1987) Nature 328:445-449. The invention also encompasses the delivery of ribozymes using the methods disclosed herein. For a discussion of suitable ribozymes, see, e.g., Cech et al., (1992) J Biol. Chem. 267:17479-17482 and U.S. Pat. No. 5,225,347.

In a preferred embodiment, rAAV virions comprising HNAs coding for one or more polypeptides are delivered to one or more cells of a secretory gland. Thus, the invention embraces the delivery of HNAs that encode one or more peptides, polypeptides, or proteins, which are useful for the treatment of disease states in a mammalian subject. Such DNA and associated disease states include, but are not limited to: DNA encoding glucose-6-phosphatase, associated with glycogen storage deficiency type 1A; DNA encoding phosphoenolpyruvate-carboxykinase, associated with Pepck deficiency; DNA encoding galactose-1 phosphate uridyl transferase, associated with galactosemia; DNA encoding phenylalanine hydroxylase, associated with phenylketonuria; DNA encoding branched chain alpha-ketoacid dehydrogenase, associated with Maple syrup urine disease; DNA encoding fumarylacetoacetate hydrolase, associated with tyrosinemia type 1; DNA encoding methylmalonyl-CoA mutase, associated with methylmalonic acidemia; DNA encoding medium chain acyl CoA dehydrogenase, associated with medium chain acetyl CoA deficiency; DNA encoding ornithine transcarbamylase, associated with ornithine transcarbamylase deficiency; DNA encoding argininosuccinic acid synthetase, associated with citrullinemia; DNA encoding low density lipoprotein receptor protein, associated with familial hypercholesterolemia; DNA encoding UDP-glucouronosyltransferase, associated with Crigler-Najjar disease; DNA encoding adenosine deaminase, associated with severe combined immunodeficiency disease; DNA encoding hypoxanthine guanine phosphoribosyl transferase, associated with Gout and Lesch-Nyan syndrome; DNA encoding biotinidase, associated with biotinidase deficiency; DNA encoding beta-glucocerebrosidase, associated with Gaucher disease; DNA encoding beta-glucuronidase, associated with Sly syndrome; DNA encoding peroxisome membrane protein 70 kDa, associated with Zellweger syndrome; DNA encoding porphobilinogen deaminase, associated with acute intermittent porphyria; DNA encoding alpha-1 antitrypsin for treatment of alpha-1 antitrypsin deficiency (emphysema); and DNA encoding a tumor suppessor gene such as p53 for the treatment of various cancers.

In an especially preferred embodiment, rAAV virions are used to deliver HNAs encoding “secretory proteins.” By “secretory proteins” is meant proteins or polypeptides that are secreted outside of the cell in which they were synthesized. Secretory proteins can be taken up by any cell (i.e., can become internally localized), including the cell in which they were synthesized, as long as they are first secreted outside of the cell in which they were synthesized. Alternatively, secretory proteins can be located to an extracellular compartment such as the extracellular matrix, the interstitial fluid, the surface of the skin, the lumen of an organ or blood vessel, or any other location not within or physically connected to a cell. By “blood vessel” is meant any vessel in the body that transports blood including, but not limited to, an artery, a vein, a venule, and a capillary.

Secretory proteins are not limited to those that are known to be naturally occurring, but encompass proteins not normally secreted in nature, which obtain the ability to be secreted by the incorporation of a signal sequence. Using well-known molecular biological techniques, the skilled artisan can insert a signal sequence in an appropriate location (usually 5′ to the start codon of a gene) within a plasmid or vector incorporating a gene, which, upon translation, enables a protein encoded therein to be secreted from the cell in which it was synthesized. Several signal sequences are known for a variety of proteins, all of which contain one or two positively charged amino acids followed generally by 6-12 hydrophobic residues (see, e.g., Leader, D. P. (1979) Trends Biochem. Sci. 4:205; Rapoport, T. A. (1985) Curr. Top. Membr. Transport 24:1-63).

The signal sequence allows a nascent polypeptide (i.e., protein) to insert itself into the membrane of the endoplasmic reticulum and translocate to the lumen of the ER where the signal sequence is then cleaved by signal peptidase. Once the signal sequence is cleaved within the lumen of the ER, the polypeptide is processed through the secretory pathway resulting in secretion of the polypeptide from the cell (for an in-depth discussion, see Blobel, G. (1995) Cold Spring Harb Symp Quant Biol. 60:1-10).

The invention encompasses DNA encoding secretory proteins that include, but are not limited to, erythropoietin for treatment of anemia due to thalassemia or to renal failure; DNA encoding vascular endothelial growth factor, DNA encoding angiopoietin-1, and DNA encoding fibroblast growth factor for the treatment of ischemic diseases; DNA encoding tissue factor pathway inhibitor for the treatment of occluded blood vessels as seen in, for example, atherosclerosis, thrombosis, or embolisms; and DNA encoding a cytokine such as one of the various interleukins for the treatment of inflammatory and immune disorders, and cancers.

More preferably, the invention encompasses rAAV virions comprising HNAs encoding blood coagulation proteins, which proteins may be delivered, using the methods of the present invention, to the cells of a mammal having hemophilia for the treatment of hemophilia. Thus, the invention includes: delivery of the Factor IX gene to a mammal for treatment of hemophilia B, delivery of the Factor VIII gene to a mammal for treatment of hemophilia A, delivery of the Factor VII gene for treatment of Factor VII, Factor VIII, Factor IX, or Factor XI deficiencies or Glanzmann thrombasthenia, delivery of the Factor X gene for treatment of Factor X deficiency, delivery of the Factor XI gene for treatment of Factor XI deficiency, delivery of the Factor XIII gene for treatment of Factor XIII deficiency, and, delivery of the Protein C gene for treatment of Protein C deficiency. Delivery of each of the above-recited genes to the cells of a mammal is accomplished by first generating a rAAV virion comprising the gene and then administering the rAAV virion to the mammal. Thus, the invention includes rAAV virions comprising genes encoding any one of Factor IX, Factor VIII, Factor X, Factor VII, Factor XI, Factor XIII or Protein C. Methods for generating human Factor VIII constructs suitable for incorporation in recombinant AAV vectors are described in U.S. Pat. Nos. 6,200,560, and 6,221,349, both herein incorporated by reference.

Recombinant AAV virions are used to deliver HNAs to secretory glands via glandular duct systems. “Secretory glands” as used herein comprise organs and/or tissues that are specialized to secrete substances, not normally related to their metabolic needs, into extracellular spaces of the body. Examples of secretory glands include, but are not limited to, the liver, pancreas, mammary glands, sweat glands, salivary glands, kidneys, pituitary, thyroid, stomach, and other glands well known in the art. Secretory glands can be either endocrine or exocrine or both. Endocrine glands generally secrete their substances into the body, e.g., into the interstitial fluid, which allows for passive diffusion of the secreted substances into the bloodstream (i.e., in an endocrine direction), whereas exocrine glands generally secrete their substances external to the body, e.g., into the lumen of an organ or onto the surface of the skin (i.e., in an exocrine direction). These distinctions are not absolute, as there is a modest transport of exocrine-secreted proteins to the bloodstream. For example, pancreatic digestive enzymes that are predominantly secreted into the pancreatic duct are also found in the bloodstream. The primary anatomical distinction between exocrine and endocrine glands is the presence or absence of ducts: Exocrine glands contain ducts whereas endocrine glands do not. Examples of endocrine glands include the pituitary gland, the thyroid gland, and the adrenal glands. Examples of exocrine glands include the sweat glands, salivary glands, and the stomach. Examples of glands that have both an exocrine and endocrine function include the liver, pancreas, and the kidneys.

Exemplary examples of secretory glands include the salivary glands and the liver. There are six salivary glands in the human: two parotid glands, two submandibular glands, and two sublingual glands, with one of each located on each side of the jaw. Each salivary gland is connected to the oral cavity by a duct or ducts, the parotid gland secreting its contents into the mouth via the parotid duct, the submandibular gland secreting its contents into the oral cavity via the submandibular duct, and the sublingual gland secreting its contents into the submandibular gland duct or the oral cavity via several small ducts.

The liver contains numerous bile ducts, which form from tiny passages in the liver cells that communicate with canaliculi (i.e., intercellular biliary passages or bile capillaries). These passages are small channels or spaces left between the contiguous surfaces of two cells, or in the angle where three or more liver cells meet and they are separated from the blood capillaries by at least half the width of a liver cell. The channels radiate to the circumference of the liver lobule, and open into the interlobular bile ducts, which run in the Glisson's capsule, accompanying the portal vein and hepatic artery. These join with other ducts to form two main trunks, which leave the liver at the transverse fissure, and by their union form the hepatic duct. The hepatic duct passes downward and to the right for about 4 cm., between the layers of the lesser omentum, where it is joined at an acute angle by the cystic duct, and so forms the common bile duct.

In general, the secretory apparatus of the liver consists of (1) the hepatic duct; (2) the gallbladder, which serves as a reservoir for the bile; (3) the cystic duct (i.e., the duct of the gallbladder); and (4) the common bile duct, formed by the junction of the hepatic and cystic ducts.

The invention encompasses the introduction of rAAV virions to the secretory gland by way of retrograde ductal administration. “Retrograde ductal administration” is defined herein as the administration of rAAV virions in a direction that is opposite to the normal flow of material in the duct. Introduction of rAAV virions can be by way of administration into the external orifice of the duct or through the duct wall so long as the rAAV virions are administered in such a manner as to cause the rAAV virions to travel in a direction opposite to the normal flow of material in the duct. Retrograde ductal administration can comprise a single, discontinuous administration (e.g., a single injection), or continuous administration (e.g., perfusion).

The invention permits the use of art-recognized non-invasive procedures to deliver rAAV virions to the secretory cells of a secretory gland. For example, the skilled artisan can use endoscopic retrograde cholangiopancreatography (ERCP) to deliver rAAV virions to the common hepatic duct of the mammalian subject. In this technique, an endoscope is inserted into the esophagus, directed through the gastrointestinal tract to the common bile duct, and threaded up through the common bile duct to the hepatic duct. The hepatic duct can then be cannulated and material can be introduced into the liver by way of retrograde ductal administration. If desired, the pancreatic duct and the cystic duct can be occluded for example, by balloon occlusion, to prevent the introduction of material to the pancreas or gallbladder. In the case of introducing material into the salivary glands, the duct can be cannulated through its orifice in the mouth and material introduced to the salivary gland by way of retrograde ductal administration.

The dose of rAAV virions required to be delivered to the secretory cells of a secretory gland to achieve a particular therapeutic effect, e.g., the units of dose in viral genomes(vg)/per mammal or vg/kilogram of body weight (vg/kg), will vary based on several factors including: the level of HNA expression required to achieve a therapeutic effect, the specific disease or disorder being treated, a potential host immune response to the rAAV virion, a host immune response to the gene product, and the stability of the gene product. In the context of dose, the term “viral genome” is synonymous with “virion,” as a viral genome comprises the rAAV vector (containing the HNA that is delivered to and transcribed in the mammal), the rAAV vector being encapsulated in the rAAV virion. When speaking of dose, viral genome is the preferred term as quantitative measurements for dose have as their endpoint the detection of viral genomes. Several such quantitative measurements are well known in the art including, but not limited to, the dot blot hybridization method (described in U.S. Pat. No. 6,335,011, herein incorporated by reference) and the quantitative polymerase chain reaction (QPCR) method (described in Real Time Quantitative PCR. Heid C. A., Stevens J., Livak K. J., and Williams P. M. 1996. Genome Research 6:986-994. Cold Spring Harbor Laboratory Press). One of skill in the art can readily determine a rAAV virion dose range to treat a patient having a particular disease or disorder based on the aforementioned factors, as well as other factors that are well known in the art.

Generally speaking, by “therapeutic effect” is meant a level of expression (i.e., “therapeutically effective levels”) of one or more HNAs sufficient to alter a component of a disease (or disorder) toward a desired outcome or clinical endpoint, such that a patient's disease or disorder shows clinical improvement, often reflected by the amelioration of a clinical sign or symptom relating to the disease or disorder. Using hemophilia as a specific disease example, a therapeutic effect for hemophilia is defined herein as an increase in the blood-clotting efficiency of a mammal afflicted with hemophilia, efficiency being determined, for example, by well known endpoints or techniques such as employing assays to measure whole blood clotting time or activated prothromboplastin time. Reductions in either whole blood clotting time or activated prothromboplastin time are indications of an increase in blood-clotting efficiency. In severe cases of hemophilia, hemophiliacs having less than 1% of normal levels of Factor VIII or Factor IX have a whole blood clotting time of greater than 60 minutes as compared to approximately 10 minutes for non-hemophiliacs. expression of 1% or greater of Factor VIII or Factor IX has been shown to reduce whole blood clotting time in animal models of hemophilia, so achieving a circulating Factor VIII or Factor IX plasma concentration of greater than 1% is considered therapeutic.

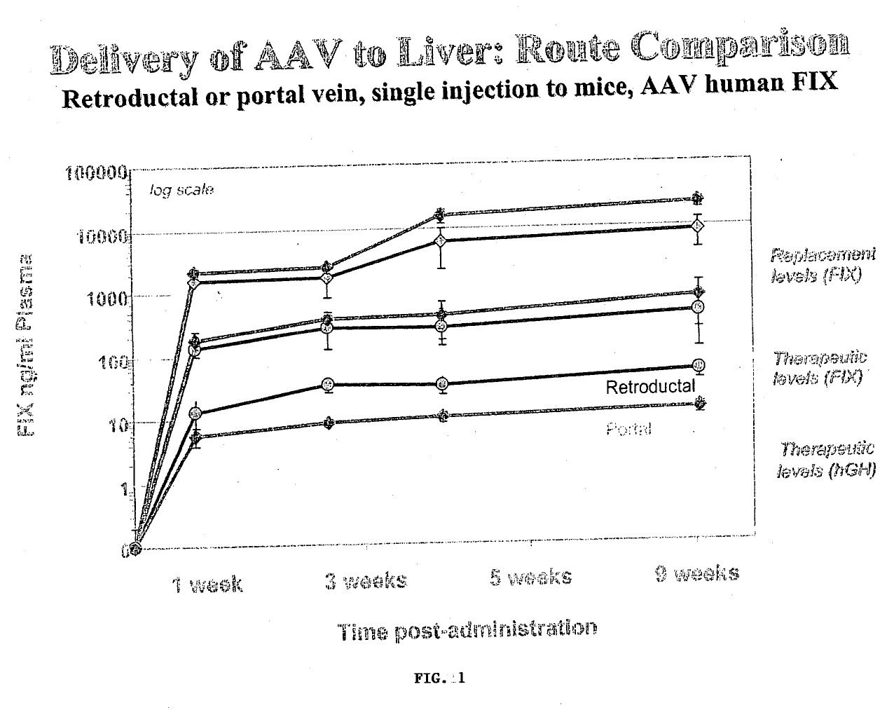

By using the methods of the present invention, rAAV virions demonstrated a high level of efficiency in transducing mouse secretory gland cells, as measured by circulating plasma levels of human Factor IX (hF.IX). As shown in FIG. 1, long-term expression of hF.IX was achieved after a single injection of rAAV-hF.IX into the submandibular gland duct of C57BI/6 naïve mice. This was true for all three dose levels. After three weeks post-transduction, serum hF.IX levels were 0.5 mg/mL for the low dose, 7.3 mg/mL for the medium dose, and 25 mg/mL for the high dose. Fifty ng/mL of serum hF.IX is generally recognized as a therapeutic level for humans, and corresponds to approximately 1% serum F.IX concentration. After nine weeks post-transduction, serum levels of hF.IX were 2 ng/mL for the low dose, 11 ng/mL for the medium dose, and 89 ng/mL for the high dose. Table 1 summarizes the serum hF.IX data.

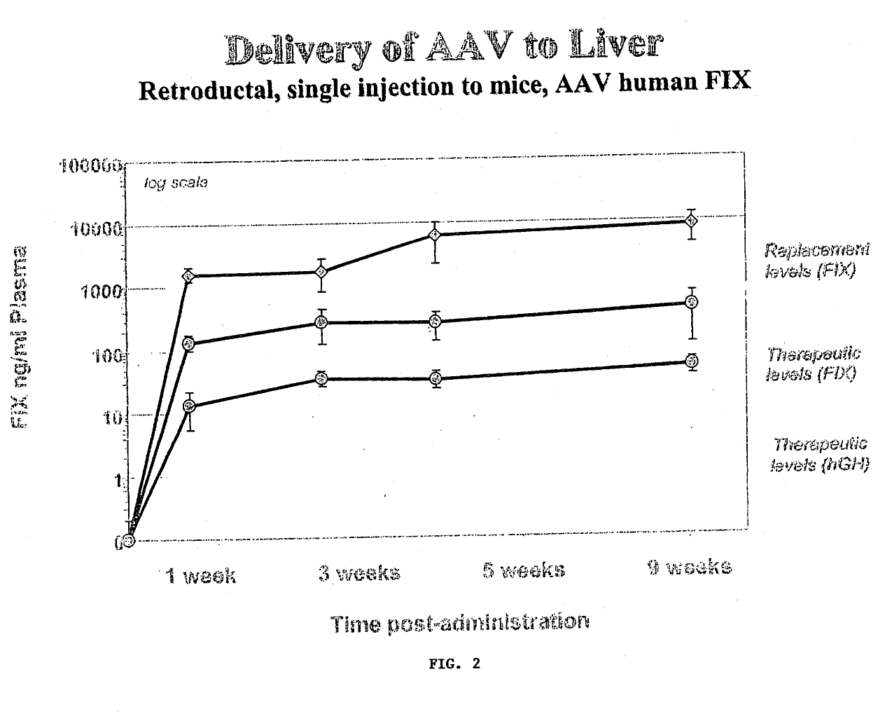

FIG. 2 depicts the results of retrograde administration of rAAV-hF.IX virions into the hepatic duct of C57BI/6 naïve mice. All three doses resulted in sustained levels of circulating hF.IX; surprisingly, expression levels for the high dose reached supraphysiological levels after five weeks post-transduction (5,985 ng/mL). After one week post-transduction, circulating levels of hF.IX reached 13 ng/mL for the low dose, 136 ng/mL for the medium dose, and 1,557 ng/mL for the high dose. Levels increased to 34 ng/mL for the low dose, 276 ng/mL for the medium dose, and 1,710 ng/mL for the high dose after three weeks post-transduction. At nine weeks post-transduction, the low dose yielded 49 ng/mL, the medium dose 426 ng/mL, and the high dose 8,365 ng/mL (see Table 1).

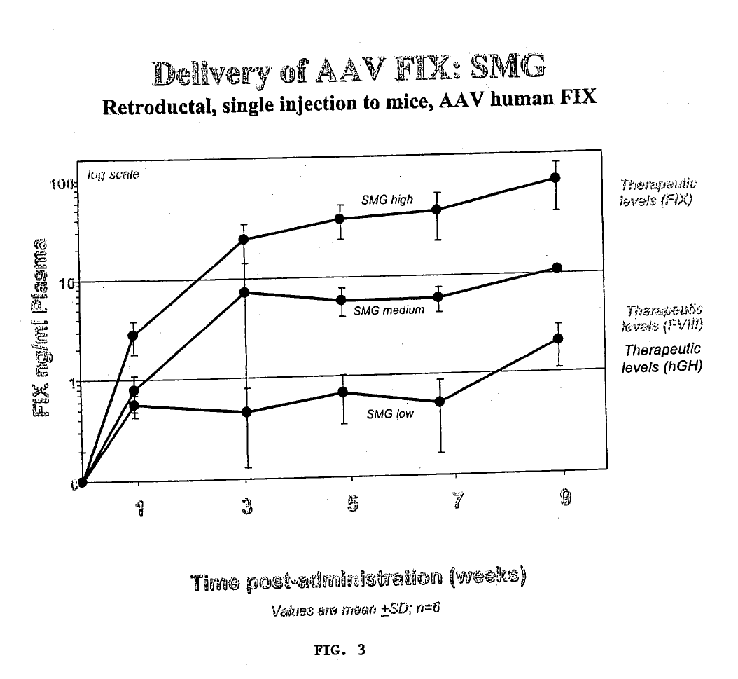

As can be seen in FIG. 3, in the low and medium dose groups, retrograde ductal administration of rAAV2-hF.IX resulted in serum levels of hF.IX that were approximately equal to those achieved by portal vein administration. In the low dose group, retrograde ductal administration was actually more efficient than portal vein administration. Low dose retrograde ductal delivery resulted in serum hF.IX levels of 13 ng/nL one week after transduction. After three weeks post-transduction, serum hF.IX levels increased to 34 ng/mL and, after nine weeks post-transduction, serum hF.IX levels increased to 49 ng/mL. For portal vein administration, the low dose group yielded serum hF.IX levels of 6 ng/mL, which increased to 8 ng/mL after three weeks, and achieved 12 ng/mL after nine weeks post-transduction (see Table 1).

The medium dose yielded similar results for both delivery methods. Only at the high dose did the portal vein delivery method achieve substantially higher concentrations of serum hF.IX than those produced by the retrograde ductal delivery method. Mice were injected intramuscularly using methods well known in the art (e.g., those described in detail in U.S. Pat. No. 5,858,351, herein incorporated by reference).

Intramuscular (i.m.) injection of mice with rAAV-hF.IX was shown to be less efficient in generating circulating titers of hF.IX (see Table 1) when compared to retrograde ductal administration into the submandibular gland or into the liver. At the high dose, i.m. injection of rAAV-hF.IX only yielded 20% of the circulating hF.IX generated by retrograde injection into the submandibular gland duct. This difference was much more dramatic in the case of the liver, as i.m. injection yielded only 0.2% of the circulating hF.IX levels generated by retrograde injection into the hepatic duct.

TABLE 1
Serum Human Factor IX Levels in C57BI/6 Naïve Mice (ng/mL)

| rAAV | | | | | |
|---|---|---|---|---|---|
| Delivery | | | | | |
| Route | | | | | |
| (dose) | 1 week* | 3 weeks | 5 weeks | 7 weeks | 9 weeks |
| Hepatic | 1,557.33 | 1,710.10 | 5,985.36 | ND | 8,364.72 |
| Duct | | | | | |
| (high) | | | | | |
| Hepatic | 135.63 | 276.35 | 258.37 | ND | 425.64 |
| Duct | | | | | |
| (med.) | | | | | |
| Hepatic | 13.37 | 33.53 | 31.93 | ND | 49.14 |
| Duct | | | | | |
| (low) | | | | | |
| SMG | 2.81 | 24.85 | 38.55 | 45.23 | 88.98 |
| (high) | | | | | |
| SMG | 0.78 | 7.28 | 5.83 | 5.90 | 10.82 |
| (med.) | | | | | |
| SMG | 0.56 | 0.46 | 0.68 | 0.52 | 2.04 |
| (low) | | | | | |
| Portal | 2,200.43 | 2,488.13 | 15,236.39 | ND | 21,7224.37 |
| Vein | | | | | |
| (high) | | | | | |
| Portal | 183.53 | 372.20 | 412.32 | ND | 754.35 |
| Vein | | | | | |
| (med.) | | | | | |
| Portal | 5.76 | 8.35 | 10.02 | ND | 12.54 |
| Vein | | | | | |
| (low) | | | | | |
| I.M. | 0.60 | 7.37 | 9.82 | 9.73 | 18.45 |
| (high) | | | | | |
| Neg. | 0.02 | 0.33 | 0.08 | 0.01 | 0.12 |
| Control** | | | | | |

*= Time post-transduction of rAAV-hF.IX.
**= rAAV-LacZ used as a negative control in SMG and hepatic duct administration.

It is often desirable to deliver an HNA to a host cell in order to elucidate its physiological or biochemical function(s). The HNA can be either an endogenous gene or a heterologous gene. Using the methods of the instant invention, the skilled artisan can administer rAAV virions containing one or more HNAs of unknown function to an experimental animal, express the HNA(s), and observe any subsequent functional changes. Such changes can include: protein-protein interactions, alterations in biochemical pathways, alterations in the physiological functioning of cells, tissues, organs, or organ systems, and/or the stimulation or silencing of gene expression.

Alternatively, the skilled artisan can over-express a gene of known function and examine its effects. Such genes can be either endogenous to the experimental animal or heterologous in nature (i.e., a transgene).

By using the methods of the present invention, the skilled artisan can also abolish or significantly reduce gene expression, thereby employing another means of determining gene function. One method of accomplishing this is by way of administering antisense RNA-containing rAAV virions to an experimental animal, expressing the antisense RNA molecule so that the targeted endogenous gene is “knocked out,” and then observing any subsequent physiological or biochemical changes.

The methods of the present invention are compatible with other well-known technologies such as transgenic mice and knockout mice and can be used to complement these technologies. One skilled in the art can readily determine combinations of known technologies with the methods of the present invention to obtain useful information on gene function.

Once delivered, in many instances it is not enough to simply express the HNA; instead, it is often desirable to vary the levels of HNA expression. Varying HNA expression levels, which varies the dose of the HNA expression product, is frequently useful in acquiring and/or refining functional information on the HNA. This can be accomplished, for example, by incorporating a heterologous inducible promoter into the rAAV virion containing the HNA so that the HNA will be expressed only when the promoter is induced. Some inducible promoters can also provide the capability for refining HNA expression levels; that is, varying the concentration of inducer will fine-tune the concentration of HNA expression product. This is sometimes more useful than having an “on-off” system (i.e., any amount of inducer will provide the same level of HNA expression product, an “all or none” response). Numerous examples of inducible promoters are known in the art including the ecdysone promoter, steroid promoters (e.g., estrogen and androgen promoters) and metallothionein promoters.

The methods of the present invention can be used to facilitate pharmaco- or toxico-kinetic studies. For example, because AAV is known to transduce hepatocytes with high efficiency, human metabolic enzymes (e.g., various oxidases and reductases such as the cytochrome p450 isozymes, various epoxide hydrolases, various dehydrogenases such as alcohol and aldehyde dehydrogenases, various peptidases, etc.—metabolic enzymes that are expressed and function in hepatocytes) can be delivered to the liver of mice by way of rAAV virions, expressed, and then various drugs and/or toxicants can be administered to the transduced mice in order to screen for any metabolites of interest.

The following examples are presented in order to more fully illustrate the preferred embodiments of the invention. They should in no way be construed, however, as limiting the broad scope of the invention, which is solely limited by the appended claims.

### EXAMPLE 1

### Recombinant AAV Factor Ix Virion Preparation

Recombinant AAV virions containing the human Factor IX (hF.IX) gene—the complete cDNA sequence for hF.IX available under GenBank Accession No. 182612—were prepared using a triple-transfection procedure described in U.S. Pat. No. 6,001,650, supra.

### Vector Construction

### AAV pHLP19 Helper Function Vector Construction

The AAV pHLP19 helper function vector was constructed using standard molecular biological techniques; its construction is described in detail in U.S. Pat. No. 6,001,650, supra.

To summarize, the AAV pHLP19 helper function vector was constructed in a several-step process using AAV-2 sequences derived from the AAV-2 provirus, pSM620, GenBank Accession Numbers K01624 and K01625. First, the ITRs were removed from the rep and cap sequences. Plasmid pSM620 was digested with SmaI and PvuII, and the 4543 bp rep-and cap-encoding SmaI fragment was cloned into the SmaI site of pUC19 to produce the 7705-bp plasmid, pUCrepcap. The remaining ITR sequence flanking the rep and cap genes was then deleted by oligonucleotide-directed mutagenesis using the oligonucleotides 145A (5′-GCTCGGTACCCGGGCGGAGGGGTGGAGTCG-3′) and 145B (5′-TAATCATTAACTACAGCCCGGGGATCCTCT-3′). The resulting plasmid, pUCRepCapMutated (PUCRCM) (7559 bp) contains the entire AAV-2 genome (AAV-2 genome, GenBank Accession Number NC_{—}001401) without any ITR sequence (4389 bp). SrfI sites, in part introduced by the mutagenic oligonucleotides, flank the rep and cap genes in this construct. The AAV sequences correspond to AAV-2 positions 146-4,534.

Second, an Eco47111 restriction enzyme site was introduced at the 3′ border of p5. This Eco47III site was introduced at the 3′ end of the p5 promoter in order to facilitate excision of the p5 promoter sequences. To do this, pUCRCM was mutagenized with primer P547 (5′-GGTTTGAACGAGCGCTCGCCATGC-3′). The resulting 7559 bp plasmid was called pUCRCM47III.

Third, an assembly plasmid, called pBluntscript, was constructed. The polylinker of pBSII SK+ was changed by excision of the original with BssHII and replaced with oligonucleotides blunt 1 and 2. The resulting plasmid, pBluntscript, is 2830 bp in length, and the new polylinker encodes the restriction sites EcoRV, HpaI, SrfI, PmeI, and Eco47III. The blunt 1 sequence is 5′-CGCGCCGATATCGTTAACGCCCGGGCGTTTAAACAGCGCTGG-3′ and the blunt 2 sequence is 5′-CGCGCCAGCGCTGTTTAAACGCCCGGGCGTTAACGATATCG G-3′.

Fourth, the plasmid pH1 was constructed by ligating the 4397 bp rep-and cap-encoding SmaI fragment from pUCRCM into the SrfI site of pBluntscript, such that the HpaI site was proximal to the rep gene. Plasmid pH1 is 7228 bp in length.

Fifth, the plasmid pH2 was constructed. Plasmid pH2 is identical to pH1 except that the p5 promoter of pH1 was replaced by the 5′ untranslated region of pGN1909 (ATCC Accession Number 69871. Plasmid pGN1909 construction is described in detail in U.S. Pat. No. 5,622,856, herein incorporated by reference in its entirety). To accomplish this, the 329 bp AscI(blunt)-SfiI fragment encoding the 5′ untranslated region from pW1909lacZ (described in detail in U.S. Pat. No. 5,622,856, supra) was ligated into the 6831 bp SmaI(partial)-SfiI fragment of pH1, creating pH2. Plasmid pH2 is 7155 bp in length.

Sixth, pH8 was constructed. A p5 promoter was added to the 3′ end of pH2 by insertion of the 172 bp, SmaI-Eco47III fragment encoding the p5 promoter from pUCRCM47III into the Eco47III site in pH2. This fragment was oriented such that the direction of transcription of all three AAV promoters are the same. This construct is 7327 bp in length.

Seventh, the AAV helper function vector pHLP19 was constructed. The TATA box of the 3′ p5 (AAV-2 positions 255-261, sequence TATTTAA) was eliminated by changing the sequence to GGGGGGG using the mutagenic oligonucleotide 5DIVE2 (5′-TGTGGTCACGCTGGGGGG GGGGGCCCGAGTGAGCACG-3′). The resulting construct, pHLP19, is 7327 bp in length.

### pLadeno5 Accessory Function Vector

The accessory function vector pLadeno5 was constructed as follows: DNA fragments encoding the E2a, E4, and VA RNA regions isolated from purified adenovirus serotype-2 DNA (obtained from Gibco/BRL) were ligated into a plasmid called pAmpscript. The pAmpscript plasmid was assembled as follows: oligonucleotide-directed mutagenesis was used to eliminate a 623-bp region including the polylinker and alpha complementation expression cassette from pBSII s/k+ (obtained from Stratagene), and replaced with an EcoRV site. The sequence of the mutagenic oligo used on the oligonucleotide-directed mutagenesis was 5′-CCGCTACAGGGCGCGATATCAGCTCACTCAA-3′. A polylinker (containing the following restriction sites: Bam HI; KpnI; SrfI; XbaI; ClaI; Bst11071; SalI; PmeI; and NdeI) was synthesized and inserted into the EcoRV site created above such that the BamHI side of the linker was proximal to the fl origin in the modified plasmid to provide the pAmpscript plasmid. The sequence of the polylinker was 5′-GGATCCGGTACCGCCCGGGCTCTAGAATCGATGTATACGTCGACGTTTAAACCAT ATG-3′.

DNA fragments comprising the adenovirus serotype-2 E2a and VA RNA sequences were cloned directly into pAmpscript. In particular, a 5962-bp SrfI-KpnI (partial) fragment containing the E2a region was cloned between the SrfI and KpnI sites of pAmpscript. The 5962-bp fragment comprises base pairs 21,606-27,568 of the adenovirus serotype-2 genome. The complete sequence of the adenovirus serotype-2 genome is accessible under GenBank No. 9626158.

The DNA comprising the adenovirus serotype-2 E4 sequences had to be modified before it could be inserted into the pAmpscript polylinker. Specifically, PCR mutagenesis was used to replace the E4 proximal, adenoviral terminal repeat with a SrfI site. The location of this SrfI site is equivalent to base pairs 35,836-35,844 of the adenovirus serotype-2 genome. The sequences of the oligonucleotides used in the mutagenesis were: 5′-AGAGGCCCGGGCGTTTTAGGGCGGAGTAACTTGC-3′ and 5′-ACATACCCGCAGGCGTAGAGAC-3′. A 3,192 bp E4 fragment, produced by cleaving the above-described modified E4 gene with SrfI and SpeI, was ligated between the SrfI and XbaI sites of pAmpscript, which already contained the E2a and VA RNA sequences to result in the pLadeno5 plasmid. The 3,192-bp fragment is equivalent to base pairs 32,644-35,836 of the adenovirus serotype-2 genome.

### Recombinant AAV2-hF.IX Vector for Submandibular Gland Delivery

The rAAV-2 hF.IX vector used for the submandibular gland (SMG) delivery was constructed using standard molecular biological techniques. It is an 11,442-bp plasmid containing the cytomegalovirus (CMV) immediate early promoter, exon 1 of hF.IX, a 1.4-kb fragment of hF.IX intron 1, exons 2-8 of h.FIX, 227 bp of h.FIX 3′ UTR, and the SV40 late polyadenylation sequence between the two AAV-2 inverted terminal repeats (details of which are contained in U.S. Pat. No. 6,093,392, herein incorporated by reference). The 1.4-kb fragment of hF.IX intron 1 consists of the 5′ end of intron 1 up to nucleotide 1098 and the sequence from nucleotide 5882 extending to the junction with exon 2. The CMV immediate early promoter and the SV40 late polyadenylation signal sequences can be obtained from the published sequence of pCMV-Script®, which is available from the Stratagene catalog, Stratagene, La Jolla, Calif., and from their website, www.stratagene.com.

### Recombinant AAV2-ApoE-HCR-hAAT-hF.IXmg-bpA Vector for Liver Delivery

In lieu of the hF.IX expression vector used for SMG delivery, a new hF.IX expression vector, created from the hF.IX cassette ApoE-HCR-hAAT-hF.IXmg-bpA, described in Miao et al., (2000) Molecular Therapy 1:522-532, was made in order to maximize expression in hepatocytes. The vector consists of the apolipoprotein E locus control region/human α1-antitrypsin promoter cassette (ApoE-HCR-hAAT) operably linked to a hF.IX gene, including a portion of the first intron (intron A), 3′-untranslated region (FIXmg), which is operably linked to a bovine growth hormone polyadenylation signal (bpA). The hF.IX gene is identical to the one used in SMG delivery, which is described above.

The cassette was constructed as follows: The apolipoprotein E locus control region (ApoE HCR, described in Schachter et al., supra) and having the sequence 5′-GCTGTTTGTGTGCTGCCTCTGAAGTCCACACTGAACAAACTTCAGCCTACTCATG TCCCTAAAATGGGCAAACATTGCAAGCAGCAAACAGCAAACACACAGCCCTCCC TGCCTGCTGACCTTGGAGCTGGGGCAGAGGTCAGAGACCTCTCTG-3′, the human alpha 1-antitrypsin (HAAT) promoter (the entire sequence published in GenBank, Accession No. D38257), the 402 bp fragment hAAT promoter sequence (described in Le et al. (1997) Blood 89:1254-1259) used in the rAAV2-ApoE-HCR-hAAT-hF.IX mg-bpA vector spans—347 to +56 of the hAAT gene (complete sequence published in GenBank, Accession No. K02212), the human F.IX minigene (described above), and the bovine growth hormone polyadenylation (bpA) sequence (sequence published in GenBank, Accession No. AF034386) were ligated into a pBluescript® backbone (Stratagene, La Jolla, Calif.) using standard molecular biological techniques, creating plasmid pBS-ApoE-HCR-hAAT-hF.IX mg-bpA.

The cassette was then excised from the pBluescript® backbone and cloned into a vector containing two AAV-2 inverted terminal repeats (ITRs) creating the recombinant AAV vector rAAV2-ApoE-HCR-hAAT-hF.IXmg-bpA, using standard molecular biological techniques. The sequence for the left ITR of AAV-2 is published under GenBank Accession No. K01624 and the right ITR sequence of AAV-2 is published under GenBank Accession No. K01625.

### Triple Transfection Procedure

Recombinant AAV2-hF.IX virions were produced using the AAV helper function pHLP19 vector, the accessory function vector pLadeno5, the rAAV2-hF.IX vector for SMG delivery (or the rAAV2-ApoE-HCR-hAAT-hF.IXmg-bpA vector for liver delivery) were used. Briefly, human embryonic kidney cells type 293 (293 cells—available from the American Type Culture Collection, catalog number CRL-1573) were seeded in 10 cm tissue culture-treated sterile dishes at a density of 3×10⁶ cells per dish in 10 mL of cell culture medium consisting of Dulbeco's modified Eagle's medium supplemented with 10% fetal calf serum and incubated in a humidified environment at 37° C. in 5% CO₂. After overnight incubation, 293 cells were approximately eighty-percent confluent. The 293 cells were then transfected with DNA by the calcium phosphate precipitate method. Briefly, 10 μg of each vector (pHLP10, pLadeno5, and rAAV2-hF.IX (or rAAV2 -ApoE-HCR-hF.IX-bpA)) were added to a 3-mL sterile, polystyrene snap cap tube using sterile pipette tips. 1.0 mL of 300 mM CaCl₂ (JRH grade) was added to each tube and mixed by pipetting up and down. An equal volume of 2× HBS (274 mM NaCl, 10 mM KCl, 42 mM HEPES, 1.4 mM Na₂PO₄, 12 mM dextrose, pH 7.05, JRH grade) was added with a 2-mL pipette, and the solution was pipetted up and down three times. The DNA mixture was immediately added to the 293 cells, one drop at a time, evenly throughout the dish. The cells were then incubated in a humidified environment at 37° C. in 5% CO₂ for six hours. A granular precipitate was visible in the transfected cell cultures. After six hours, the DNA mixture was removed from the cells, which were then provided with fresh cell culture medium and incubated for an additional 72 hours.

After 72 hours, the cells were lysed and then treated with nuclease to reduce residual cellular and plasmid DNA. After precipitation, rAAV virions were purified by two cycles of isopycnic centrifugation; fractions containing rAAV virions were pooled, dialysed, and concentrated. The concentrated virions were formulated, sterile filtered (0.22 μM) and aseptically filled into glass vials. Viral genomes were quantified by the “Real Time Quantitative Polymerase Chain Reaction” method (Real Time Quantitative PCR. Heid C. A., Stevens J., Livak K. J., and Williams P. M. 1996. Genome Research 6:986-994. Cold Spring Harbor Laboratory Press).

### EXAMPLE 2

### Retrograde Ductal Administration into the Submandibular Gland of Mice

C57BI/6 naïve mice were divided into three dose groups, 6 animals per group, and injected with 50 μL of rAAV-hF.IX viral genomes in three doses: 1×10⁹ rAAV-hF.IX viral genomes comprising the low dose, 1×10¹⁰ rAAV-hF.IX viral genomes comprising the medium dose, and 1×10¹¹ rAAV-hF.IX viral genomes comprising the high dose. The mice were anesthetized and an incision made in the inner cheek to expose the duct of the submandibular gland. Recombinant AAV-hF.IX virions were injected into the duct of the submandibular gland in a retrograde direction.

Circulating hF.IX levels were measured in mouse plasma using ELISA, as described in U.S. Pat. No. 6,093,392, herein incorporated by reference, and in Walter et al. (1996) Proc. Natl. Acad. Sci. 93:3056-3061. As the primary antibody, a polyclonal rabbit anti-human F.IX antibody was used in a dilution of 1:1200. Mouse plasma samples were diluted in buffer (PBS/0.05% Tween/6% BSA) to concentrations within the range of the standard curve. A polyclonal goat anti-human F.IX antibody coupled to horseradish peroxidase was used as the secondary antibody in a dilution of 1:500. Table 1 and FIG. 1 depict levels of circulating hF.IX.

### EXAMPLE 3

### Retrograde Ductal Administration into the Liver of Mice

C57BI6/naïve mice were infused with 250 μL of rAAV-hF.IX virions via retrograde ductal administration to the hepatic duct. Mice were anesthetized and, under an operating microscope, a midline abdominal incision was conducted to gain access to the cystic duct. The falciform ligamentum anterior was separated and the median liver lobe was displaced to expose the gallbladder, cystic duct, hepatic duct, and the common bile duct. The common bile duct was clamped off above the juncture with the pancreatic duct to prevent anterograde flow of vector to the duodenum and retrograde flow to the pancreas. Prior to clamping the common bile duct, however, the common bile duct was flushed with saline. Silk suture was placed loosely around the proximal site of the gallbladder and the cystic duct was cannulated. Recombinant AAV virions were slowly infused into the cannula. Three dose groups were established, with 6 mice per dose group, the low dose group receiving 1×10⁹ rAAV-hF.IX viral genomes, the medium dose group receiving 1×10¹⁰ rAAV-h.FIX viral genomes, and the high dose group receiving 1×10¹¹ rAAV-hF.IX viral genomes. After infusion, the distal end of the polyethylene tube was coagulated, all retractors and the xyphoid clamp relieved, and the intestinal duct placed back in its original position. One hour after rAAV virion infusion, the anterograde flow from the bile duct to the duodenum was restored by removing the clamp from the common bile duct. The abdomen was then closed in two layers. Circulating hF.IX levels were measured as in Example 2. Table 1 and FIG. 2 depict levels of circulating hF.IX.

### EXAMPLE 4

### Portal Vein Administration

C57BI/6 naïve mice were separated into three dose groups, 3 mice per group and injected with 1×10⁹ rAAV-hF.IX viral genomes (low dose), 1×10¹⁰ rAAV-hF.IX viral genomes (medium dose), and 1×10¹¹ rAAV-hF.IX viral genomes (high dose). Mice were injected with rAAV virions into the portal vein according to the procedures described in Nakai et al. (1998) Blood 91:4600-4607. In adult mice, rAAV-hF.IX virions were administered into the portal circulation through an injection beneath the splenic capsule. Animals were anesthetized and the portal vein was exposed through a ventral midline incision followed by displacement of the intestinal duct. Recombinant AAV virion solution was slowly injected into the portal vein with a Hamilton syringe. The peritoneal cavity was sutured and the skin closed. Circulating hF.IX levels were measured as in Example 2. Table 1 and FIG. 3 depict levels of circulating hF.IX.

What is claimed is:

1. A method of delivering a protein to a mammal, comprising:
a) providing recombinant adeno-associated virus (rAAV) virions, wherein said rAAV virions are free of helper virus, and wherein said rAAV virions comprise a heterologous gene encoding a protein;
b) contacting said rAAV virions with a duct of a secretory gland of said mammal wherein said contacting results in transduction of at least one cell of said secretory gland;
c) expressing said heterologous gene; and
d) secreting said protein.

2. The method of claim 1, wherein said secreting of said protein results in a therapeutic effect.

3. The method of claim 2, wherein said protein is a blood coagulation protein.

4. The method of claim 3, wherein said blood coagulation protein is Factor IX.

5. The method of claim 4, wherein said Factor IX is human Factor IX.

6. The method of claim 3, wherein said blood coagulation protein is Factor VIII.

7. The method of claim 6, wherein said Factor VIII is human Factor VIII.

8. The method of claim 1, wherein said rAAV virion is delivered to said duct of said secretory gland by retrograde ductal administration.

9. The method of claim 1, wherein said secretory gland is a salivary gland.

10. The method of claim 9, wherein said salivary gland is a submandibular gland.

11. The method of claim 1, wherein said secretory gland is a liver.

12. The method of claim 11, wherein said rAAV virion is delivered to said liver by endoscopic retrograde cholangiopancreatography.

13. The method of claim 11, wherein said bile duct is a hepatic duct.

14. The method of claim 12, wherein said bile duct is a hepatic duct.

15. The method of claim 11, wherein said bile duct is a common bile duct.

16. The method of claim 12, wherein said bile duct is a common bile duct.

17. The method of claim 1, wherein said mammal is a human.

18. A method of delivering human Factor IX to a mammal, comprising:
a) providing recombinant adeno-associated virus (rAAV) virions at a dose from about 1×10⁹ to about 1×10¹¹ rAAV viral genomes, wherein said rAAV virions are free of helper virus, and wherein said rAAV virions comprise a heterologous gene encoding human Factor IX;
(b) contacting said rAAV virions with a duct of a salivary gland of said mammal wherein said contacting results in transduction of at least one cell of said duct of said salivary gland;
(c) expressing said heterologous gene; and
(d) secreting said human Factor IX into a blood vessel of said mammal.

19. A method of delivering human Factor IX to a mammal, comprising:
a) providing recombinant adeno-associated virus (rAAV) virions at a dose from about 1×10⁹to about 1×10¹¹ rAAV viral genomes, wherein said rAAV virions are free of helper virus, and wherein said rAAV virions comprise a heterologous gene encoding human Factor IX;
(b) contacting said rAAV virions with a duct of a liver of said mammal wherein said contacting results in transduction of at least one cell of said duct of said liver;
(c) expressing said heterologous gene; and
(d) secreting said human Factor IX into a blood vessel of said mammal wherein said human Factor IX is present in said blood vessel at a therapeutic level.

20. A method of treating hemophilia in a mammal, comprising:
a) providing recombinant adeno-associated virus (rAAV) virions, wherein said rAAV virions are free of helper virus, and wherein said rAAV virions comprise a heterologous gene encoding a blood coagulation protein;
b) contacting said rAAV virions with a duct of a secretory gland of said mammal wherein said contacting results in transduction of at least one cell of said secretory gland;
c) expressing said heterologous gene; and
d) secreting said blood coagulation factor into a blood vessel of said mammal wherein therapeutically effective levels of said blood coagulation factor are achieved.

21. The method of claim 20, wherein said hemophilia is hemophilia B.

22. The method of claim 20, wherein said hemophilia is hemophilia A.

23. The method of claim 20, wherein said blood coagulation factor is Factor VIII.

24. The method of claim 23, wherein said Factor VIII is human Factor VIII.

25. The method of claim 20, wherein said coagulation factor is Factor IX.

26. The method of claim 25, wherein said Factor IX is human Factor IX.

27. The method of claim 20, wherein said rAAV virion is delivered to said duct of said secretory gland by retrograde ductal administration.

28. The method of claim 20, wherein said secretory gland is a salivary gland.

29. The method of claim 28, wherein said salivary gland is a submandibular gland.

30. The method of claim 20, wherein said secretory gland is a liver.

31. The method of claim 30, wherein said rAAV virion is delivered to said liver by endoscopic retrograde cholangiopancreatography.

32. The method of claim 20, wherein said expression control element is a tissue-specific promoter.

33. The method of claim 32, wherein said tissue-specific promoter is a liver-specific promoter.

34. The method of claim 33, wherein said liver-specific promoter is a human α₁-antitrypsin promoter.

35. The method of claim 33, wherein said liver-specific promoter is operably linked to an apolipoprotein E hepatic control region.

36. The method of claim 20, wherein said mammal is a human.
